(19) United States
(12) Patent Application Publication
(10) Pub. No.: US 2012/0276539 A1
(43) Pub. Date: Nov. 1, 2012

(54) USE OF METASTASIS PROGRESSOR S100A4 TRANSCRIPTS IN BODY FLUIDS OF COLORECTAL AND GASTRIC CANCER PATIENTS

(75) Inventors: Ulrike Stein, Panketal (DE); Pia Herrmann, Berlin (DE); Susen Burock, Berlin (DE); Peter Michael Schlag, Berlin (DE)

(73) Assignee: MAX-DELBRUECK-CENTRUM FÜR MOLEKULARE MEDIZIN, Berlin (DE)

(21) Appl. No.: 13/509,460

(22) PCT Filed: Mar. 19, 2010
(86) PCT No.: PCT/EP2010/001891

§ 371 Date: Jul. 15, 2012

Related U.S. Application Data
(60) Provisional application No. 61/261,017, filed on Nov. 13, 2009.

(51) Int. Cl. C12Q 1/68

(52) U.S. Cl. 435/6.11; 435/6.12; 435/6.14

(57) ABSTRACT

Transcript quantification of the metastasis progressor S100A4 is performed in body fluids. Methods, assays and kits relying on this quantification are disclosed that have diagnostic and/or prognostic applications in colorectal and gastric cancer patients.

### FIELD OF THE INVENTION

The invention is directed at a method, assay and/or kit for transcript quantification of the metastasis-promoting gene S100A4 in a subject and correlating the levels of transcript so quantified to S100A4 transcript levels in relevant controls, for the diagnosis and/or prognosis of cancers, in particular gastrointestinal cancers.

### INTRODUCTION AND BACKGROUND OF THE INVENTION

Gastrointestinal cancers such as colon, rectal, and gastric cancers account for those malignancies with the highest incidences and mortalities worldwide.

The publications and other materials, including patents, used herein to illustrate the invention and, in particular, to provide additional details respecting the practice are incorporated herein by reference. For convenience, the publications are referenced in the following text by a number and/or are listed the appended bibliography.

About 90% of all cancer deaths arise from the metastatic dissemination of primary tumors (1, 2).

To date, clinical outcome parameters, such as local recurrence and distant metastasis of primary colon carcinomas can not be sufficiently differentiated via conventional clinical and histopathological/immunohistochemical examination: The tumor-node-metastasis (TNM) classification, represents the main tool for identifying prognostic differences among patients with early-stage colorectal cancer (3); Gastric cancer is currently evaluated by histological staging as well (4).

Therefore, there is a need for early detection of tumors and metastasis, which is critical for improving treatment strategies and the patient's recovery. There is in particular a clear need for molecular markers and for simple tests that can be clinically used for detection, prognostication, and therapy monitoring of cancer. A non-invasive blood based test for early identification of high risk cancer patients is of particular interest.

### SUMMARY OF THE INVENTION

Generally, the invention is directed at a method, assay and/or kit for transcript quantification of the metastasis-promoting gene S100A4 in a subject, in particular a human subject (“target subject”). For this quantification, all that is required is a sample of a body fluid, in particular a blood sample, of the target subject. The method, assay and/or kit is generally simple, reliable and/or can be performed quickly. If reference values, such as the basal S100A4 transcript value, are provided, the time required to carry out the method, assay and/or kit corresponds roughly to the time required to perform the transcript quantification. The method, assay and/or kit can be used for newly diagnosed and/or already treated patients with colorectal and gastric cancer as well as for general cancer screening. The method, assay and/or kit may also be used prognostically for metastasis formation and disease-free survival (DFS) for follow-up patients. Patients that can thus be classified as, e.g., high-risk for metastasis via the method, assay and/or kit of the present invention, can be more closely observed and/or treatment regimes can be devised to counteract this risk. The method, assay and/or kit thus allows for monitoring of disease progression, therapy efficacy and/or response.

The quantitative S100A4 transcript determination in body fluids such as plasma allows for routine clinical application. For example, a blood sample can be taken daily or weekly from a target subject and the method, kit and/or assay allows determination of S100A4 transcript level in the target subject at, e.g., daily or weekly intervals as well as a correlation to the S100A4 transcript levels of relevant cohorts or populations. The method allows in certain embodiments, early defining cancer staging and/or risk for metastasis in colorectal and/or gastric cancer patients.

The invention is in particular directed to a method for diagnosis and/or prognosis of a cancer, in particular a gastrointestinal cancer, a stage of gastrointestinal cancer or a gastrointestinal cancer free stage comprising:

(a) providing a basal transcript level or a transcript level of a stage of said cancer, in particular gastrointestinal cancer;
(b) measuring a S100A4 transcript level in a body fluid of a target subject;
making a determination as to whether or whether not there is a significant difference in S100A4 transcript level between (a) and (b); and
making a diagnosis and/or prognosis as to whether or whether not the target subject suffers from a cancer, in particular a gastrointestinal cancer or a certain stage thereof or is likely to have reached a cancer, in particular gastrointestinal cancer, free stage based on said determination.

Transcript levels of two or more stages of cancer, in particular gastrointestinal cancer may be provided in (a).

The diagnosis and/or prognosis of the target subject may include whether or not the target subject has one of said two or more stages of cancer, in particular gastrointestinal cancer. The determination as to whether or not there is a significant differences in S100A4 transcript level may comprise or consist essentially of comparing the values obtained for (a) with that measured in (b).

The invention is also directed to a method for the diagnosis and/or prognosis of a gastrointestinal cancer comprising:

providing a basal S100A4 transcript level;
measuring a S100A4 transcript level in a body fluid of a target subject;
making a determination as to whether or whether not there is a significant difference in S100A4 transcript level between the base S100A4 transcript level and said S100A4 transcript level in said target subject; and
making a diagnosis and/or prognosis as to whether or whether not the target subject suffers from a gastrointestinal cancer based on said determination.

RT-PCR may be used for said measuring of said S100A4 transcript level in said target subject.

Said basal S100A4 transcription level may be provided from measurement, preferably measurements obtained via RT-PCR, of one or more previous cohorts of tumor fee subjects.

Said basal transcript level may provided for one or more m RNA calibrators used for said measurements in said one or more previous cohorts of tumor free subjects.

Said basal S100A4 transcript level may be provided from measurements of a contemporary population of the target subject.

The target subject may be a patient newly diagnosed with a primary tumor; a patient newly diagnosed with a primary tumor together with synchronous metastases; a patient who already underwent R0-surgery of a primary cancer and was newly diagnosed with metachronous metastases; a follow-up patient without metastases or a follow-up patient with metastases.

The invention is also directed at a method for evaluating and/or diagnosing a target subject for a metastases of a colorectal cancer comprising:

providing a S100A4 transcript level representative of colorectal cancer patients having a primary tumor without a metastasis;
providing a S100A4 transcript level representative of colorectal cancer patients having primary tumors with synchronous or metachronous metastases;
measuring S100A4 transcript level in a body fluid of a target subject having colorectal cancer and a primary tumor;
making a determination as to whether or whether not there is a significant difference in S100A4 transcript level between the S100A4 transcript level in a target subject and the S100A4 transcript level representative of colorectal cancer patients having a primary tumor without a metastasis and/or the S100A4 transcript level representative of colorectal cancer patients having a primary tumor with synchronous or metachronous metastases,
evaluating and/or diagnosing as to whether or whether not the target subject suffers from a synchronous or metachronous metastasis based on said determination.

The metastasis of the target subject may be a synchronous metastasis and said target subject may have colon or rectal cancer and a primary tumor.

The metastasis of the target subject may be a metachronous metastasis and said target subject may have colon cancer and a primary tumor.

RT-PCR may be used for said measuring of S100A4 transcript level in a target subject having colorectal cancer and a primary tumor.

Said S100A4 transcript level representative of colorectal cancer patients having primary tumors without a metastasis and/or said S100A4 transcript level representative of colorectal cancer patients having primary tumors and synchronous or metachronous metastases, may be provided from measurements, preferably measurements obtained via RT-PCR, of one or more previous cohorts of colorectal cancer patients having primary tumors without metastasis or having primary tumors with synchronous or metachronous metastases, respectively.

The transcript level for said colorectal cancer patients having primary tumor without metastases and/or for said colorectal cancer patients having primary tumors with synchronous or metachronous metastases may be provided for one or more mRNA calibrators used for said measurements in said one or more previous cohorts.

Said S100A4 transcript level representative of colorectal cancer patients having a primary tumor without metastasis and/or said S100A4 transcript level representative of colorectal cancer patients having primary tumor and a synchronous or metachronous metastasis may be provided from measurement provided in a contemporary population of the target subject.

The invention is also directed at a method for making a prognosis of a metastasis of a gastrointestinal cancer comprising:

providing a S100A4 transcript level representative of gastrointestinal cancer patients without metastases;
providing a S100A4 transcript level representative of gastrointestinal cancer patients with metastases;
measuring S100A4 transcript level in a body fluid of a target subject wherein said target subject is a follow-up patient;
making a determination whether or whether not there is a significant difference in S100A4 transcript level between the S100A4 transcript level in said target subject and the S100A4 transcript level representative of gastrointestinal cancer patients with metastases and the S100A4 transcript level representative of gastrointestinal cancer patients without metastases,
making a prognosis as to whether or whether not the target subject will suffer from a gastrointestinal cancer metastasis based on said determination.

RT-PCR may be used for said measuring said S100A4 transcript level in said target subject.

Said S100A4 transcript level representative of gastrointestinal cancer patients without metastasis and/or said S100A4 transcript level representative of gastrointestinal cancer patients with metastases, may be provided from measurements, preferably measurements obtained via RT-PCR, of one or more previous cohorts of gastrointestinal cancer patients without metastasis and with metastases, respectively.

The transcript level for gastrointestinal cancer patients without metastasis and/or for gastrointestinal cancer patients with metastases may be provided for one or more mRNA calibrators used for said measurements in said one or more previous cohorts.

Said S100A4 transcript level representative of gastrointestinal cancer patients without metastasis and/or said S100A4 transcript level representative of gastrointestinal cancer patients with metastasis may be provided from measurement provided in a contemporary population of the target subjects.

The invention is also directed to a method for making a prognosis of a gastrointestinal cancer free state in a target subject comprising:

providing a S100A4 transcript level representative of follow-up gastrointestinal cancer patients or a basal S100 A4 transcript level;
measuring S100A4 transcript level in a body fluid of a target subject wherein said target subject is a follow-up gastrointestinal cancer patient;
making a determination as to whether or whether not there is a significant difference in S100A4 transcript level between the S100A4 transcript level in the target subject and the S100A4 transcript level representative of gastrointestinal cancer follow-up patients or the basal S100A4 transcript level,
making the prognosis as to whether or whether not the target subject will maintain a free state based on said determination.
RT-PCR may be used for said measuring S100A4 transcript level in a target subject.

Said S100A4 transcript level representative of the S100A4 transcript level in the target subject or the S100A4 transcript level representative of gastrointestinal cancer follow-up patients or the basal S100A4 transcript level may be provided from measurements, preferably measurements obtained via RT-PCR, of one or more previous cohorts of follow-up gastrointestinal cancer patients or tumor free subjects, respectively.

The S100A4 transcript level for gastrointestinal cancer patients without metastasis or the basal S100A4 transcript level may be provided for one or more mRNA calibrators used for said measurements in one or more previous cohorts.

Said S100A4 transcript level representative of follow-up gastrointestinal cancer patients or tumor free subjects may be provided from measurement provided in a contemporary population of the target subjects.

The invention is also directed at a kit for transcript quantification of S100A4 in a target subject comprising, in different containers:
a tool for quantitatively determining levels of S100A4 transcripts in a body fluid; and
values for a basal S100A4 transcript levels or instructions of how to obtain a basal transcription level of S100A4 transcripts.

The kit may further comprise a mRNA calibrator for the S100A4 transcripts in said body fluid, which in one embodiment corresponds to the calibrator used to measure provided values for the basal transcript levels.

Said tool may also comprise:

(a) primers for reverse transcribing S100A4 transcripts in a sample;
(b) a reverse transcriptase for producing cDNA of said S100A4 transcripts;
(c) DNA primers for amplifying said cDNA, and
(d) a DNA polymerase for extending said primers.

The kit may further include values or instruction of how to obtain values for a transcription level representative of different stages of gastrointestinal cancers, in particular representative for patients newly diagnosed with a primary tumor, for patients newly diagnosed with a primary tumor together with synchronous metastases; for patients who already underwent R0-surgery of the primary cancer and were newly diagnosed with metachronous metastases; for follow-up patients without metastases; for follow-up patients with metastases; follow-up gastrointestinal cancer patients with or without metastases or any combination of the same and preferably a calibrator that were used in obtaining said values.

The values may be provided as part of the kit.

The instructions may include information as to where those values can be obtained.

The kit may further comprise instructions of what values need to be or can be compared for the prognosis and/or diagnosis of gastrointestinal cancer, its stages and/or a gastrointestinal cancer free stage.

### DETAILED DESCRIPTION OF VARIOUS AND PREFERRED EMBODIMENTS OF THE INVENTION

### Definitions

A “body fluid” according to the present invention is in particular blood, plasma, any blood based or plasma based substance such as serum or any other body fluid or substance based thereon in which RNA levels can be measured, preferably using RT-PCR, and are representative for the levels in blood and/or plasma. Other body fluids include, but are not limited to, saliva or urine.

In the context of the present invention, any method that allows for quantitative RNA measurements, in particular the measurement of mRNA, may be used. RT-PCR (quantitative real-time RT-PCR) refers to a technique to amplify RNA. The RNA is first reverse transcribed into its DNA complement via, e.g., reverse transcriptase, and the resulting cDNA is amplified using real time PCR. The cDNA so measured is an accurate reflection of the amount of RNA originally present in a sample. If the present invention refers to “measuring transcript level via RT-PCR”, this cDNA mediated measurement is meant. The RT step can be performed either in the same tube with PCR (one-step PCR) or in a separate one (two-step PCR) using a temperature which is generally between 40° C. and 65° C., depending on the properties of the reverse transcriptase used.

Any microarray that comprises the gene for S100A4 and allows for the quantitative measurement of S100A4 expression can also be used to quantify the mRNA.

“Basal transcript level” (or basal transcription level) of a gene, such as S100A4, is the transcription level observed in tumor free subjects, preferably human subjects. The basal transcription level is generally determined relative to a certain calibrator. Any construct that expresses S100A4 including cell lines, vectors and plasmids can be used as such a calibrator and is within the scope of the present invention.

Transcription level, such as basal transcript levels, obtained from measurements of a “contemporary population” relative to a target subject means that the transcription levels are taken within 12, 11, 10, 9, 8, 7, 6, 5, 4, 3, 2 or 1 months from the measurements within the same geographical area the target subject stems from, such as a region, city or town.

Transcription level, such as basal transcript levels, obtained from measurements of one or more “previous cohorts” relative to a target subject means that the transcription levels has been established previously, generally temporarily and/or spacially remote from the target subject. Such transcription levels can be looked up online either publically or via a code provided or are available in publications, such as brochures, package inserts or are generally available publications.

A “target subject” is a subject that either may or may not suffer from a gastrointestinal cancer, currently is suffering from a gastrointestinal cancer and is or is not currently being treated or has been treated for cancer such as gastrointestinal cancer.

“Gastrointestinal cancers” according to the present invention include in particular, but are not limited to, colorectal cancers including colon and rectal cancers as well as gastric cancers. Gastrointestinal cancer patients can be classified into different stages: patients newly diagnosed with a primary tumor; patients newly diagnosed with a primary tumor together with synchronous metastases; patients who already underwent R0-surgery of the primary cancer, generally somewhere else, and were newly diagnosed with metachronous metastases; and follow-up patients without metastases or follow-up patients with metastases. Follow-up patients underwent R0-surgery of the primary cancer, generally in the same institution and were further observed/monitored for metastases development after the determination of S100A4 transcripts in a body fluid.

“Significant differences in S100A4 transcripts” according to the present invention are transcript levels in a body fluid, in particular in a human body fluid, in particular blood, plasma or serum, that have a P<0.05 according to the non-parametric Wilcoxon-Mann-Whitney test.

DFS (disease free survival) is defined as survival, free of events of tumor, recurrence or metastases formation. Generally, a patient cohort is observed ranging from at least two years to at least three years, at least 4 years, at least 5 years or at least 6 years or more. In the context of gastrointestinal cancers, DFS is also referred to as a gastrointestinal cancer free state.

Blood-based diagnostics are not only useful for snap-shots, like tumor marker determination in patients biopsies, but allow monitoring of disease progression, therapy efficacy and response. Cancer diagnostics with plasma-based expression profiling is quick, relatively simple and cost-effective, and particularly attractive from the point of view of patient comfort. Circulating nucleic acids, and in particular cell-free mRNA can be detected in plasma and permits plasma-based expression profiling (5-9). Garcia et al. reported that (10) the quantitative detection of tumor-derived transcripts in blood might allow the identification of occult tumors and metastases in apparently healthy individuals. However, no reliable non-invasive blood-based methods are routinely used for the detection and grading of cancer.

S100A4 (metastasin, mts1, see also U.S. Pat. No. 5,798,257) plays an important role as metastasis progressor. It is a member of the multigene S100 family of calcium-binding proteins. Intracellular S100A4 interacts with proteins of the cytoskeleton, such as actin filaments, non-muscle tropomyosin, non-muscle myosin II, thereby directly increasing cell motility (11-13). S100A4 also induces a migratory phenotype by affecting cell adhesion via binding to liprin 131(14). Moreover, S100A4 binds to the tumor suppressor p53 and modulates its transcriptional activity (15). Extracellular S100A4 activates expression of several matrix metalloproteinases thereby enabling cell invasion into adjacent tissues and facilitating angiogenesis (16, 17). High levels of S100A4 correlate with reduced patient survival and poor prognosis in several types of cancer, such as, but not limited to, thyroid, epithelial, lung, liver, kidney, breast, lymphoid, hematopoietic, pancreatic, endometrial, ovarian, cervical, skin, colorectal and gastric cancer (18-21). In colorectal cancer, high S100A4 expressions correlate with aggressive tumor growth and poor prognosis (22-28). Overexpression of S100A4 was also related to aggressiveness and metastasis in gastric cancer (29-32).

It was previously shown that S100A4 transcripts when quantitatively determined in the non-metastasized primary tumor have potential value for prognosis of metastasis formation in colon cancer patients (29). Previous attempts to use S100A4 as a tumor marker in plasma and serum, failed in view of the release of S100A4 from normal blood cells (24).

S100A4 was found to act as metastasis progressor for several human cancers, and is prognostic for metachronous metastasis when determined in the solid primary tumor. High levels of S100A4 correlate with reduced patient survival and poor prognosis. Particularly in colorectal and gastric cancer, high S100A4 expressions correlate with aggressive tumor growth and poor prognosis. Here we provide for the first time a blood-based assay for transcript quantification of S100A4 in patients' plasma, that would allow clinical application routinely. We demonstrated its diagnostic potential in plasma of colorectal and gastric cancer patients, even for early defining cancer staging. The prognostic value of S100A4 transcripts for patients' risk for metastasis and for disease-free survival is shown for follow-up patients. Our data on metastasis progressor S100A4 mRNA—that now can be monitored in patients' plasma—bears direct impact on diagnosis and prognosis of colorectal and gastric cancer patients, and might contribute to personalized therapy.

### Materials and Methods

Patients, blood, and plasma

Patients suffering from colon, rectal, and gastric cancer, who were treated at the Robert-Rossle-Cancer Hospital, Charité University Medicine Berlin, during a 14 months period for the first or for a further time, were enrolled (Table 1). Histological tumor staging was performed by routine pathology.

Patients data describing the histopathological characterization of the tumor (including tumor infiltration, lymph node status, distant metastasis, grading, lymphatic vessels infiltration, blood vessels infiltration, residual tumor), documentation of the therapy, and data on survival were available from the tumor bank. Patients were classified into the following groups:
patients who were newly diagnosed with a primary tumor of the colon (UICC stage I: T1-2N0M0, II: T3-4N0M0, III: T1-4N0-1M0), the rectum (I: T1-2N0M0, II: T3-4N0M0, III: T1-4N0-1M0), or the stomach (I: T1-2N0-1M0, II: T1-3N0-2M0, III: T2-4N0-2M0, IV: T1-4N1-3M0);
patients who were newly diagnosed with a primary colon, rectal (both IV: T1-4N0-1M1), or gastric tumor (IV: T4N1-3M1), together with synchronous distant metastases;
patients who already underwent R0-surgery of primary colon, rectal, or gastric cancer, and were newly diagnosed with metachronous distant metastases. In addition, colorectal cancer patients were grouped in accordance to their chronological examination and initial blood taking for evaluation of the diagnostic value of S100A4 transcript determination (Table 2):
those patients (with tumor, with tumor and synchronous metastasis, and with metachronous metastasis) who were analyzed first constituted the so-called test set (n=44) for calculation of the optimal S100A4 mRNA cut-off with regard to sensitivity and specificity;
those patients who were examined in the following constituted the so-called validation set (n=43), and were evaluated based on this cut-off.

The cohort of gastric cancer patients was not split because of the number of samples (n=27).

Furthermore, we included follow-up patients who visited the physician for follow-up examinations after colon, rectal, or gastric cancer R0-surgery and were diagnosed to be tumor-free and metastasis-free, or who developed metastases during follow-up (blood samples were taken in median 1726, 1055, and 944 days after primary diagnosis). For a comprehensive list of all patients and their characteristics see Table 1.

Patients were not treated with chemotherapy or surgery within the last two weeks prior to taking the blood sample. The period between the end of the treatment and taking the blood sample was about 3-6 days for locally advanced rectal cancer patients treated with neoadjuvant short-course radiation. Surgery was performed within one week for the short-course patients after the end of the treatment, but within 4-6 weeks for chemoradiation patients.

Patients who had a primary tumor of another entity in history or who developed a second primary tumor during follow-up were excluded from this analysis. Several studies report the involvement of S100A4 in non-malignant diseases such as inflammation and heart diseases (33-35). Therefore, patients who suffered from active inflammation processes, from coronary heart disease or who had recently a heart attack were also excluded.

Altogether, 466 blood samples of tumor patients were examined, with 185, 190, and 91 samples of colon, rectal, and gastric cancer patients, respectively. Blood was collected daily from hospitalized patients and from the outpatient care. Blood specimens were obtained with informed written consent. In accordance to the International Conference on Harmonization patients' anonymity and confidentiality are preserved. Additionally, blood samples of 51 tumor-free volunteers, collected in two independent cohorts, were analyzed. The whole procedure has been approved by the local IRB.

Plasma was separated as described by Fleischhacker et al (36). Briefly, 5 ml of cooled EDTA-blood were centrifuged at 1300 rpm for 10 minutes and 10° C. The plasma supernatant was again centrifuged at 2500 rpm for 15 minutes and 4° C. to remove all cell debris. Plasma samples were stored at −80° C. Samples were blinded, so that neither tumor entity nor disease stage were disclosed during analysis.

### RNA and Quantitative Real Time RT-PCR

Total RNA was isolated from plasma by using the High Pure Viral RNA kit according to the recommendations of the manufacturer (ROCHE DIAGNOSTICS). RNA quality was initially controlled by capillary gel electrophoresis (2100 Bioanalyzer, AGILENT). Quantitative, two step, gene-specific, real time RT-PCR was carried out as previously described (ROCHE DIAGNOSTICS) (27). For PCR, the following primer and probes were employed to generate the 124 bp amplicon of S100A4: forward-primer 5′-gagctgcccagcttcttg-3′, reverse-primer 5′-tgcaggacaggaagacacag-3′, FITC-probe 5′-tgatgagcaacttggacagcaaca-3′, LCRed640-probe 5′-gacaacgaggtggacttccaagagt-3′. Each sample was run in duplicate. Calibration was performed with a standard curve, carried along in each run.

### Statistical Analysis

Results are expressed as median (range) or frequencies (%). After proof of the distribution for normality, differences between the regarded groups in terms of S100A4 transcript levels in plasma were tested by using non-parametric Wilcoxon-Mann-Whitney tests, e.g. comparing tumor-free volunteers to patients with tumors, to patients harboring tumors and synchronous metastases, to patients with metachronous metastases, and to follow-up patients without or with metastases. Likewise, comparison of S100A4 in plasma of patients with tumors vs. those with tumors and metastases, or for follow-up patients without vs. with metastases, were carried out by means of this test. In case of small samples, greater differences in sample sizes, large but unbalanced groups, data sets containing ties, or sparse data, tests were carried out in an exact version. We considered P<0.05 to be significant. Accounts for multiple comparisons were carried out using the sequentially rejective Bonferroni-Holm procedure (37). All numerical calculations were performed with SPSS, version 16.

To define the diagnostic value of S100A4 transcripts in plasma, a cut-off value of S100A4 sensitivity and specitity was calculated with a fourfold table for colorectal cancer (test and validation set), rectal cancer, colon cancer and gastric cancer patients, who were newly diagnosed with a primary tumor without or with synchronous metastases, and for patients who already underwent R0 (resection for cure)-surgery of the primary cancer and were newly diagnosed with metachronous metastases compared to the blood samples of 51 tumor-free volunteers.

For DFS (disease free survival) in all follow-up patients, Kaplan Meier curves in combination with log rank test were used. The cut-off value of S100A4 was the median of S100A4 of the entire follow-up groups (metastasized together with non-metastasized) of each entity. These calculations were performed with SPSS, version 17.

### Results

### S100A4 Transcript Levels in Tumor-Free Volunteers

First, we defined the basal S100A4 transcript level in human plasma. We analyzed blood of two independent cohorts of tumor-free volunteers from Charité Campus Berlin-Buch (n=36) and from Charité Campus Virchow-Klinikum (n=15) for S100A4 (FIG. 1). S100A4 transcripts were detectable in all samples, and remained almost unchanged when determined repeatedly in monthly intervals in the same tumor-free individuals. Since the S100A4 levels were not significantly different in both cohorts (P=0.9259; cohort I: median 0.224; cohort II: median 0.203), we combined them for all subsequent analyses (median 0.219) (FIG. 1). Significant variations of S100A4 levels due to age and sex of the individuals were not observed. S100A4 levels showed neither circadian dependence nor dependence on food intake.

S100A4 Transcript Levels in Plasma Discriminate Cancer Patients from Tumor-Free Volunteers

Next, we assessed S100A4 transcript plasma levels for discrimination of cancer patients and tumor-free individuals (Table 1). We began with colorectal cancer patients' blood (n=375). S100A4 mRNA was detected in all plasma samples. We determined a statistically significant difference for all colorectal cancer patients' samples compared to the tumor-free volunteers (P<0.0001; FIG. 2A). This also holds true when subclassifying the colorectal cancer patients into a colon (n=185; P<0.0001; FIG. 3A) and a rectal cancer patients sub-cohort (n=190; P<0.0001; FIG. 3B). Additionally, we analyzed blood from gastric cancer patients and found significantly higher S100A4 transcript levels compared to the tumor-free individuals (n=91; P<0.0001; FIG. 4A).

In a next step, patients were classified due to disease stages: patients who were newly diagnosed with a primary tumor, patients who were newly diagnosed with a primary tumor together with synchronous metastases, patients who already underwent R0-surgery of the primary cancer and were newly diagnosed with metachronous metastases, and follow-up patients without or with metastases. Remarkably, colorectal (Table 1, FIG. 2A), colon (Table 1, FIG. 3A), rectal (Table 1, FIG. 3B), and gastric cancer patients (Table 1, FIG. 4A) of each of these groups demonstrated significantly higher S100A4 transcript levels than the tumor-free volunteers. No significant differences were found when comparing S100A4 levels to patients characteristics such as age and sex in any of these groups and for any of each entities.

To evaluate the diagnostic power of this blood-based S100A4 mRNA assay, we analyzed sensitivity and specificity for patients, who were newly diagnosed with a primary tumor without or with synchronous metastases, and for patients who already underwent R0-surgery of the primary cancer and were newly diagnosed with metachronous metastases. We began with a test set of colorectal cancer patients (n=44), with a sensitivity of 73% and a specificity of 82% (optimal cut-off 0.358). This cut-off was then used for the validation set of colorectal cancer patients (n=43), resulting in a sensitivity of 68% and a specificity of 82%. When combining all colorectal cancer patients, sensitivity was 71% and specificity was 82%. Then, we applied this cut-off for the sub-group of colon cancer patients (n=28) and found a sensitivity of 68% and a specificity of 82%. For rectal cancer patients the sensitivity with this cut-off was 72% and the specificity 82%. The optimal cut-off values for the colon and rectal cancer patients were 0.232 and 0.346 with sensitivity of 96% and 74%, and specificities of 59% and 82%, respectively. For gastric cancer patients (n=27), we determined a sensitivity of 90% and a specificity of 71% (cut-off 0.289) (Table 2).

S100A4 Transcript Levels in Plasma of Cancer Patients Newly Diagnosed with a Primary Tumor without or with Distant Metastases

Next, S100A4 levels of the patients' groups were compared within a defined entity. S100A4 levels were found to be independent of UICC stages within the patients' group with an primary tumor only, as observed for all entities. Increased S100A4 levels were determined for colorectal cancer patients and for the sub-cohort of colon cancer patients, suffering from a primary tumor and synchronous metastases compared to patients (UICC stage IV) with a primary tumor only (UICC stage I-III) (P=0.0472; FIG. 2A and P=0.0053; FIG. 3A, respectively). Furthermore, S100A4 levels were higher in colon cancer patients with metachronous metastases compared to those with a primary tumor only (UICC stage I-III) (P=0.0022, FIG. 3A). Thus, the determination of S100A4 transcript plasma levels point preferentially to an improved diagnosis for distant metastases of colon cancer patients. No significantly different S100A4 levels were found for metastasized rectal and gastric cancer patients, either synchronously or metachronously, compared to patients with only the primary tumor (FIGS. 3B and 4A, respectively).

S100A4 Transcript Levels in Plasma of Follow-Up Cancer Patients without or with Distant Metastases

We also analyzed S100A4 in plasma of follow-up colon, rectal, and gastric cancer patients. Although all of them underwent R0-surgery, the patients' groups of each entity showed increased S100A4 levels compared to tumor-free volunteers (Table 1). Some patients thereof developed distant metastases during the follow-up, but after the S100A4 transcript determination (median follow-up after blood taking for colon, rectal, and gastric cancer: 742, 705, and 735 days, respectively). Very interestingly, metastasized patients had demonstrated higher S100A4 levels in tendency compared to the non-metastasized ones. According to the sequentially rejective test procedure, the tests show multiple significance for all analyzed colorectal cancer patients (P=0.0111) and for patients with rectal cancer (P=0.038), with only two exceptions, patients with colon (P=0.0504) and gastric cancer (P=0.0595), comparing metastasized to non-metastasized patients. Thus, determination of the S100A4 transcript levels in plasma might be of prognostic value for metastases formation of follow-up patients with these gastrointestinal malignancies.

### S100A4 Transcript Levels in Plasma of Follow-Up Cancer Patients and DFS

We also investigated the S100A4 transcript levels in plasma of all follow-up patients in the context of DFS. DFS was defined as survival, free of the events of tumor, recurrence, and metastases formation. Interestingly, unfavourable DFS was observed for S100A4 high expressing colorectal as well as gastric cancer patients (FIGS. 2B, 4B). In particular, DFS was significantly reduced in follow-up patients with high S100A4 levels suffering from colorectal cancer (P=0.013, log-rank test; FIG. 2B). The cut-off value of S100A4 was the median of S100A4 calculated for the entire follow-up group of each entity (colorectal cancer 0.387, and gastric cancer 0.39). Thus, the transcript levels of S100A4 are not only of diagnostic, but also of prognostic value.

### Discussion

The transcript determination of the metastasis progressor S100A4 in human blood is reported and the diagnostic and prognostic power of S100A4 transcripts in blood samples from colon, rectal, and gastric cancer patients was demonstrated. These analyses are based on data including our own that demonstrate the diagnostic and prognostic value of S100A4 when detected in solid tumors (18-32). Circulating nucleic acids including transcripts in plasma or serum of patients have been evaluated as diagnostic, prognostic and predictive markers for solid neoplasias (5-10). For gastric cancer, osteopontin plasma levels have been shown to be significantly associated with invasion and patients survival (38). hTERT and MUC1 mRNAs were found to be overexpressed in plasma of gastric cancer patients, but were not detectable in plasma of healthy volunteers (39).

For colorectal cancer, hTERT(N) mRNA in plasma clearly differentiates between healthy and colorectal cancer patients (40). Elevated levels of epithelial tumor RNA (CK19, CEA) and thymidylate synthase transcripts in plasma of colon cancer patients are associated with advanced stages and poor prognosis (41-45). The prognostic value of LISCH7 transcripts, a gene that is regulated by p53 and overexpressed in colon cancer metastasis development, was shown in plasma of colon cancer patients (46).

Interestingly, increased levels of β-catenin, that plays a crucial role for Wnt pathway signalling and colorectal pathogenesis, have been determined in plasma and were correlated to tumor stage (47). Activation of the Wnt/β-catenin pathway is frequently observed in colorectal cancers, and β-catenin gene mutations have been described as early and critical steps in the genesis of the disease. We demonstrated previously that β-catenin/TCF directly regulates the expression of S100A4, and that gain-of-function β-catenin acts in a dominant manner (27, 48). Activation of Wnt/β-catenin signalling together with high S100A4 expression was also reported for gastric cancer (31, 32).

S100A4 mRNA was determined quantitatively in human plasma, and found higher S100A4 levels in cancer patients than in tumor-free volunteers, with sensitivities of 71% and 90% as well as specificities of 82% and 71% for colorectal and gastric cancer patients, respectively. Thus, S100A4 mRNA detection is applicable and might support screening for occult tumors and/or metastasis in healthy populations. The use of S100A4 protein as tumor marker in human blood, however, was critically evaluated (49). We have shown the diagnostic relevance of S100A4 transcript plasma levels even for early disease stages prior to the event of metastasis, since the S100A4 levels in patients with a non-metastasized primary tumor were higher than those of the control groups of each entity analyzed. In particular, we found significantly higher S100A4 levels in synchronously or metachronously metastasized colon cancer patients. Thus, S100A4 transcript levels might also contribute to identify a metastasized disease stage particularly for colon cancer.

So far, only 3 out of all 63 non-metastasized primary tumors did developed distant metastases during the follow-up period (median follow-up for colon, rectal, and gastric cancer: 726, 762, and 693 days, respectively). Therefore, we could not evaluate the prognostic impact of S100A4 transcripts in plasma for the individual metachronous metastasis risk for these cohorts with a primary tumor only, that have previously been shown by us and others when determined in tissues of the non-metastasized primary tumor. Longer follow-up times and higher numbers of blood samples might confirm the previous knowledge but employing the new blood-based technology.

However, we determined prospectively S100A4 transcript plasma levels in all 352 follow-up cases. Interestingly, follow-up patients who subsequently developed distant metastases expressed high S100A4 transcript levels at the initial blood taking. Thus, metastases formation correlated with high S100A4 levels in plasma of colon, rectal, and gastric cancer patients. This remarkable finding points to the prognostic value of S100A4 with respect to disease course (metastasis) for follow-up patients, who account with 74% for the largest groups of all analyzed patients. Notably, DFS was also lowered for follow-up patients with high S100A4 transcript plasma levels, particularly for colorectal cancer patients. This finding supports the role of S100A4 also for patients' survival. Moreover, it underlines the usefulness of this blood-based assay for monitoring purposes.

We also analyzed the influence of tumor-related neoadjuvant treatments on S100A4 levels in locally advanced rectal cancer. We analyzed the 40 primary rectal cancer cases (stage I-III) before and post neoadjuvant treatment. We found a clear but not significant decrease of S100A4 transcripts in the treated patients' group independent of the treatment schedule (short-course radiotherapy or chemoradiation), that might point to a dependency of S100A4 levels on therapy modalities. This would provide the rationale for a S100A4-based monitoring of therapy response. However, the diverse intervals between neoadjuvant treatment and taking the blood sample (at least 3 days for short-course radiation and at most 6 weeks for chemoradiation) might account for the non-significant decrease of S100A4 transcripts. Further studies will be necessary to elucidate the potential of S100A4 transcripts in blood for prediction and monitoring of therapy response. For colon cancer there is no recommendation of neoadjuvant treatment and the impact of neoadjuvant therapy in gastric cancer is still controversial.

In summary, we demonstrated the diagnostic and prognostic potential of S100A4 transcripts in plasma of colon, rectal, and gastric cancer patients for early defining cancer staging and patients' risk for metastasis. For this purpose, we developed a non-invasive, reliable, and simple method, that would allow clinical application routinely. Our data on metastasis progressor S100A4 mRNA—that is now detectable in patients' plasma—might also contribute to personalization of initial and additional therapy that could strongly enhance patient care. Combinatorial detection of relevant transcripts might enhance diagnosis, prognosis or even prediction. Further S100 genes such as S100A8 and S100A9, recently identified as protein markers in colorectal cancer patients' blood, or transcripts already described in the context of gastrointestinal cancer, might add to a more comprehensive and thus personalized approach (50).

### DESCRIPTION OF THE FIGURES

FIG. 1.

S100A4 transcripts in plasma of tumor-free volunteers.

Box plot analysis, based on quantitative real-time RT-PCR (Table 1). No different S100A4 transcript levels were found in two independently analyzed cohorts of tumor-free volunteers.

FIG. 2.

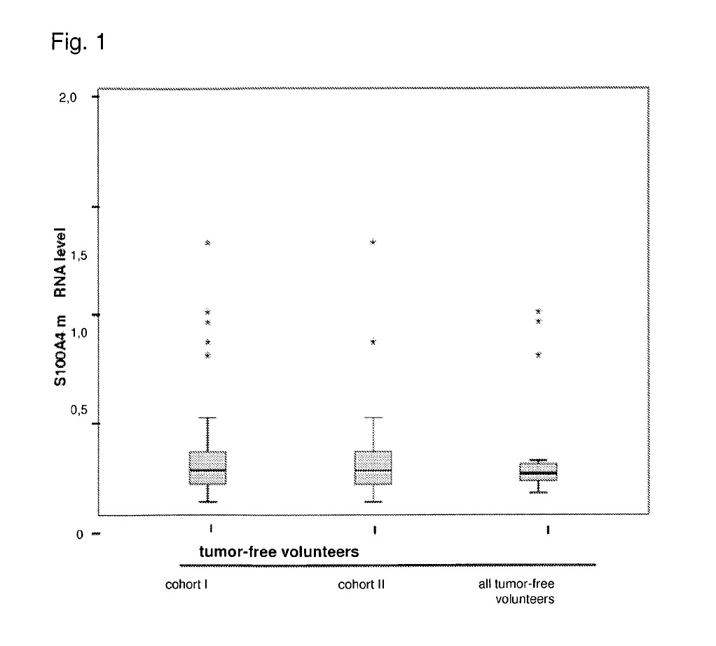

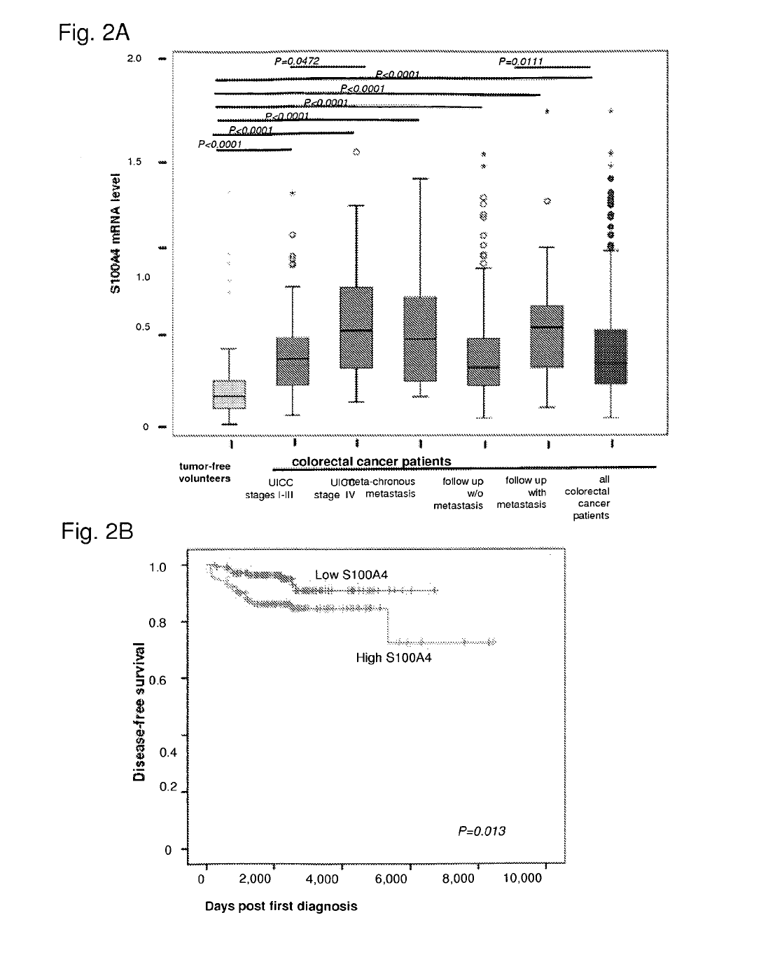

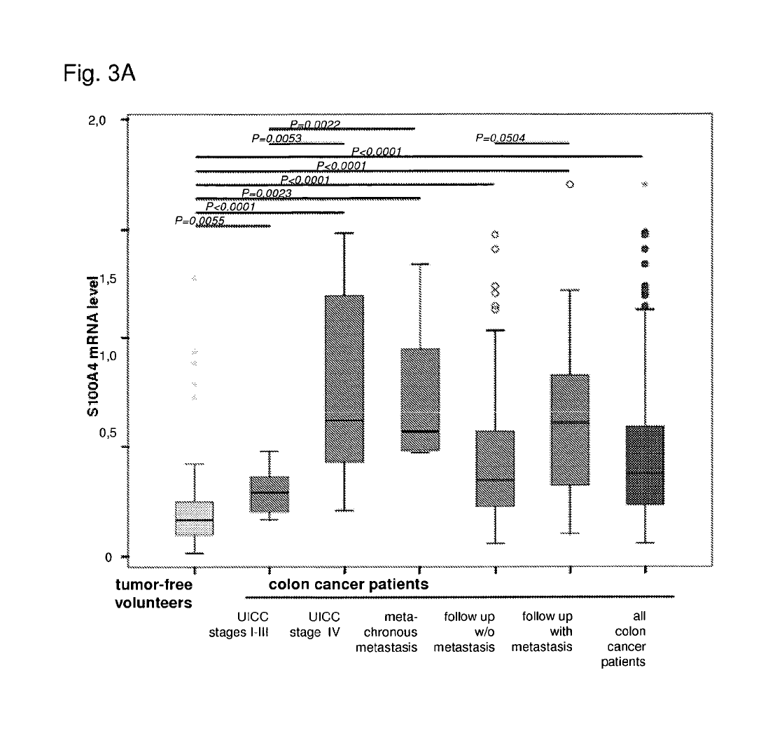

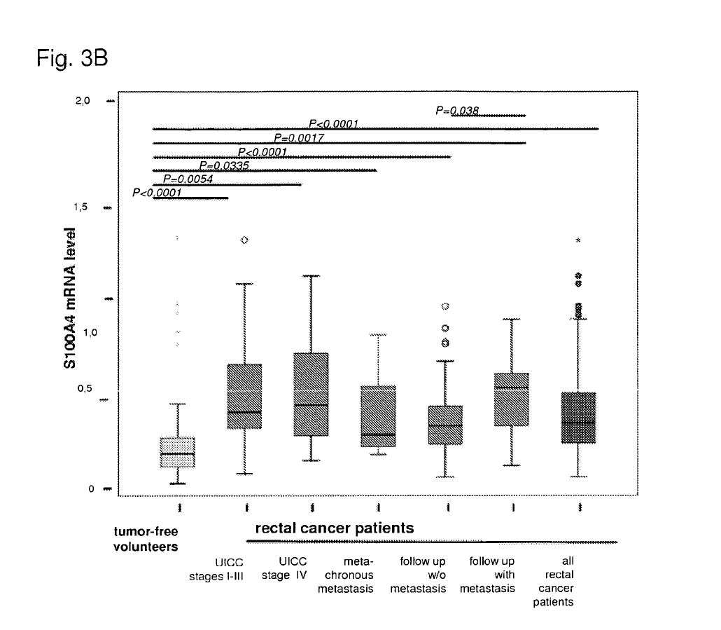

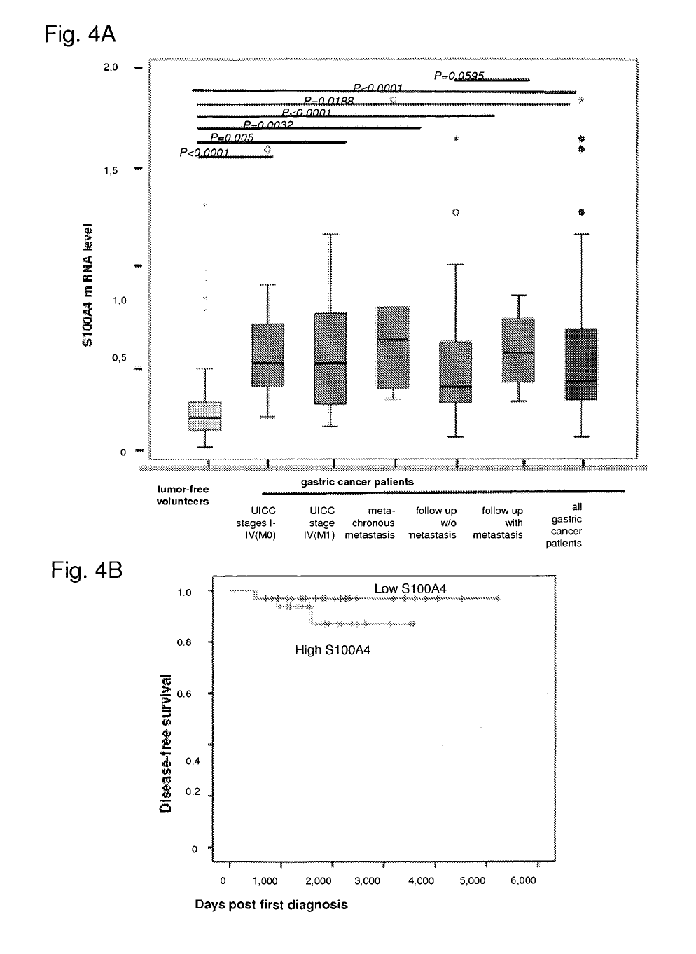

S100A4 transcripts in plasma of colorectal cancer patients.

A) Box plot analysis, based on quantitative real-time RT-PCR (Table 1). All patients' cohorts expressed significantly higher S100A4 transcripts than healthy volunteers. Different S100A4 levels were also found for patients of stages I-III vs. IV, and for follow-up patients without and with metastases.

B) Kaplan-Meier analysis for DFS of all follow-up patients (n=288, Table 1). Based on the median (0.387), all follow-up patients were sub-classified into S100A4 transcript low (below median; n=145) and high (above median; n=143) expressors. DFS was significantly decreased in patients with high S100A4 transcript levels (P=0.013).

FIG. 3.

S100A4 transcripts in plasma of colon and rectal cancer patients.

A) Box plot analysis, based on quantitative real-time RT-PCR (Table 1). All patients' cohorts expressed significantly higher S100A4 transcripts than healthy volunteers. Different S100A4 levels were also found for patients of stages I-III vs. IV or vs. metachronously metastasized patients, and for follow-up patients without and with metastases.

B) Box plot analysis, based on quantitative real-time RT-PCR (Table 1). All patients' cohorts expressed significantly higher S100A4 transcripts than healthy volunteers. Different S100A4 levels were also found for follow-up patients without and with metastasis formation.

FIG. 4.

S100A4 transcripts in plasma of gastric cancer patients.

A) Box plot analysis, based on quantitative real-time RT-PCR (Table 1). All patients' cohorts expressed significantly higher S100A4 transcripts than healthy volunteers. Different S100A4 levels were also found for follow-up patients without and with metastases.

B) Kaplan-Meier analysis for DFS of all follow-up patients (n=64, Table 1). Based on the median (0.39), all follow-up patients were sub-classified into S100A4 transcript low (below median; n=32) and high (above median; n=32) expressors.

Of course, the embodiments of the invention described above are intended to be exemplary only and shall not be construed as limiting the invention. The reader is referred to the summary or invention and the claims.

### BIBLIOGRAPHY

1. Christofori G. New signals from the invasive front. Nature 2006; 441:444-50.
2. Stein U, Schlag P M. Clinical, biological, and molecular aspects of metastasis in colorectal cancer. Recent Res Cancer Res 2007; 176:61-79.
3. Beahrs O H. Staging of cancer of the colon and rectum. Cancer 1992; 70:1393-6.
4. Hayashi H, Ochiai T, Suzuki T, et al. Superiority of a new UICC-TNM staging system for gastric carcinoma. Surgery 2000; 127:129-35.
5. Anker P, Mulcahy H, Stroun M. Circulating nucleic acids in plasma and serum as a noninvasive investigation for cancer: time for large-scale clinical studies? Int J Cancer 2003; 103:149-52.
6. El-Hefnawy T, Raja S, Kelly L, et al. Characterization of amplifiable, circulating RNA in plasma and its potential as a tool for cancer diagnostics. Clin Chem 2004; 50:564-73.
7. Goebel G, Zitt M, Zitt M, Müller H M. Circulating nucleic acids in plasma or serum (CNAPS) as prognostic and predictive markers in patients with solid neoplasias. Dis Markers 2005; 21:105-20.
8. Fleischhacker M, Schmidt B. Circulating nucleic acids (CNAs) and cancer—a survey. Biochim Biophys Acta 2007; 1775:181-232.

9. Swarup V, Rajeswari MR. Circulating (cell-free) nucleic acids—A promising, non-invasive tool for early detection of several human diseases. FEBS Lett 2007; 581:795-9.
10. Garcia J M, García V, Peña C, et al. Extracellular plasma RNA from colon cancer patients is confined in a vesicle-like structure and is mRNA-enriched. RNA 2008; 14:1424-32.
11. Takenaga K, Nakamura Y, Sakiyama S, Hasegawa Y, Sato K, Endo H. Binding of pEL98 protein, an 5100-related calcium-binding protein, to nonmuscle tropomyosin. J Cell Biol 1994; 124:757-68.
12. Chen H, Fernig D G, Rudland P S, Sparks A, Wilkinson M C, Barraclough R. Binding to intracellular targets of the metastasis-inducing protein, S100A4 (p9Ka). Biochem Biophys Res Commun 2001; 286:1212-7.
13. Li Z, Bresnick A R. The S100A4 metastasis factor regulates cellular motility via a direct interaction with myosin-IIA. Cancer Res 2006; 66:5173-80.
14. Kriajevska M, Fischer-Larsen M, Moertz E, et al. Liprin beta 1, a member of the family of LAR transmembrane tyrosine phosphatase-interacting proteins, is a new target for the metastasis-associated protein S100A4 (Mts1). J Biol Chem 2002; 277:5229-35.
15. Grigorian M, Andresen S, Tulchinsky E, et al: Tumor suppressor p53 protein is a new target for the metastasis-associated Mts1/S100A4 Protein. J Biol Chem 2001; 276:22699-708.
16. Schmidt-Hansen B, Ornas D, Grigorian M, et al. Extracellular S100A4(mts1) stimulates invasive growth of mouse endothelial cells and modulates MMP-13 matrix metalloproteinase activity. Oncogene 2004; 23:5487-95.
17. Semov A, Moreno M J, Onichtchenko A, et al. Metastasis-associated Protein S100A4 induces angiogenesis through interaction with annexin II and accelerated plasmin formation. J Biol Chem 2005; 280:20833-41.
18. Helfman D M, Kim E J, Lukanidin E, Grigorian M. The metastasis associated protein S100A4: role in tumour progression and metastasis. Br J Cancer 2005; 92:1955-8.
19. Garrett S C, Varney K M, Weber D J, Bresnick A R. S100A4, a mediator of metastasis. J Biol Chem 2006; 281:677-80.
20. Tarabykina S, Griffiths T R, Tulchinsky E, Mellon J K, Bronstein I B, Kriajevska M. Metastasis-associated protein S100A4: spotlight on its role in cell migration. Curr Cancer Drug Targets 2007; 7:217-28.
21. Sherbet G V. Metastasis promoter S100A4 is a potentially valuable molecular target for cancer therapy. Cancer Lett 2009; 280:15-30.
22. Takenaga K, Nakanishi H, Wada K, et al. Increased expression of S100A4, a metastasis-associated gene, in human colorectal adenocarcinomas. Clinical Cancer Res 1997; 3:2309-16.
23. Gongoll S, Peters G, Mengel M, et al. Prognostic significance of calcium-binding protein S100A4 in colorectal cancer. Gastroenterology 2002; 123:1478-84.
24. Flatmark K, Pedersen K B, Nesland J M, et al. Nuclear localization of the metastasis-related protein S100A4 correlates with tumour stage in colorectal cancer. J Pathol 2003; 200:589-95.
25. Cho Y G, Kim C J, Nam S W, et al. Overexpression of S100A4 is closely associated with progression of colorectal cancer. World J Gastroenterol 2005; 11:4852-6.
26. Hemandas A K, Salto-Tellez M, Maricar S H, Leong A F, Leow C K. Metastasis-associated protein S100A4—a potential prognostic marker for colorectal cancer. J Surg Oncol 2006; 93:498-503.
27. Stein U, Arlt F, Walther W, et al. The metastasis-associated gene S100A4 is a novel target of beta-catenin/T-cell factor signaling in colon cancer. Gastroenterology 2006; 131:1486-1500.
28. Kim J H, Kim C N, Kim S Y, et al. Enhanced S100A4 protein expression is clinicopathologically significant to metastatic potential and p53 dysfunction in colorectal cancer. Oncol Rep 2009; 22:41-7.
29. Yonemura Y, Endou Y, Kimura K, et al. Inverse expression of S100A4 and E-cadherin is associated with metastatic potential in gastric cancer. Clinical Cancer Res 2000; 6:4234-42.
30. Kim Y J, Kim M A, Im S A, et al. Metastasis-associated protein S100A4 and p53 predict relapse in curatively resected stage III and IV (M0) gastric cancer. Cancer Invest 2008; 26:152-8.
31. Yoon C S, Hyung W J, Lee J H, et al. Expression of S100A4, E-cadherin, alpha- and beta-catenin in gastric adenocarcinoma. Hepatogastroenterology 2008; 55:1916-20.
32. Li Y, Zhang K L, Sun Y, et al. Frequent S100A4 expression with unique splicing pattern in gastric cancers: a hypomethylation event paralleled with E-cadherin reduction and Wnt activation. Transl Oncol 2008; 1:165-76.
33. Heizmann C W, Ackermann G E, Galichet A. Pathologies involving the S100 proteins and RAGE. Subcell Biochem 2007; 45:93-138.
34. Grigorian M, Ambartsumian N, Lukanidin E. Metastasis-inducing S100A4 protein: implication in non-malignant human pathologies. Curr Mol Med 2008; 8:492-6.
35. Oslejsková L, Grigorian M, Gay S, Neidhart M, Senolt L. The metastasis associated protein S100A4: a potential novel link to inflammation and consequent aggressive behavior of rheumatoid arthritis synovial fibroblasts. Ann Rheum Dis 2008; 67:1499-1504.
36. Fleischhacker M, Beinert T, Ermitsch M, et al. Detection of amplifiable messenger RNA in the serum of patients with lung cancer. Ann N Y Acad Sci 2001; 945:179-88.
37. Holm S. A simple sequentially rejective multiple test procedure. Scand J Statistics 1979; 6:65-70.
38. Wu C Y, Wu M S, Chiang E P, et al. Elevated plasma osteopontin associated with gastric cancer development, invasion and survival. Gut 2007; 56:782-9.
39. Tani N, Ichikawa D, Ikoma D, et al. Circulating cell-free mRNA in plasma as a tumor marker for patients with primari and recurrent gastric cancer. Anticancer Res 2007; 27:1207-12.
40. Lledo S M, Garcia-Granero E, Dasi F, et al. Real time quantification in plasma of human telomerase reverse transcriptase (hTERT) mRNA in patients with colorectal cancer. Colorectal Dis 2004; 6:236-42.
41. Silva J M, Rodriguez R, Garcia J M, et al. Detection of epithelial tumour RNA in the plasma of colon cancer patients is associated with advanced stages and circulating tumour cells. Gut 2002; 50:530-4.
42. Goldstein M J, Mitchell EP. Carcinoembryonic antigen in the staging and follow-up of patients with colorectal cancer. Cancer Invest 2005; 23:338-51.
43. Garcia V, Garcia J M, Pena C, et al. Thymidylate synthase messenger RNA expression in plasma from patients with colon cancer: prognostic potential. Clinical Cancer Res 2006; 12:2095-100.
44. Wang J Y, Wu C H, Lu C Y, et al. Molecular detection of circulating tumor cells in the peripheral blood of patients with colorectal cancer using RT-PCR: significance of the prediction of postoperative metastasis. World J Surg 2006; 30:1007-13.
45. Wang J Y, Lin S R, Wu D C, et al. Multiple molecular markers as predictors of colorectal cancer in patients with normal perioperative serum carcinoembryonic antigen levels. Clinical Cancer Res 2007; 13:2406-13.
46. Garcia J M, Peña C, Garcia V, et al. Prognostic value of LISCH7 mRNA in plasma and tumor of colon cancer patients. Clinical Cancer Res 2007; 13:6351-7.
47. Wong S C, Lo S F, Cheung M T, et al. Quantification of plasma beta-catenin mRNA in colorectal cancer and adenoma patients. Clinical Cancer Res 2004; 10:1613-7.
48. Sack U, Stein U. Wnt up your mind—intervention strategies for S100A4-induced metastasis in colon cancer. Gen Physiol Biophys (in press) 2009
49. Flatmark K, Maelandsmo G M, Mikalsen S O, et al. Immunofluorometric assay for the metastasis-related protein S100A4: release of S100A4 from normal blood cells prohibits the use of S100A4 as a tumor marker in plasma and serum. Tumour Biol 2004; 25:31-40.
50. Kim H J, Kang H J, Lee H, et al. Identification of S100A8 and S100A9 as serological markers for colorectal cancer. J Proteome Res 2009; 8:1368-79.

TABLE 1
Characteristics of colorectal, colon, rectal, and gastric cancer patients, and S100A4 transcript levels
Colorectal cancer patients characteristics and S100A4 transcript levels

| | | colorectal cancer patients | | | | | |
|---|---|---|---|---|---|---|---|
| | | UICC | UICC | meta- | follow-up | follow-up | |
| | tumor-free | stages | stage | chronous | w/o | with | all cancer |
| | volunteers | I-III | IV | metastasis | metastasis | metastasis | patients |
| Donor characteristics | | | | | | | |
| samples, n | 51 | 53 | 21 | 13 | 261 | 27 | 375 |
| UICC I, % | | 43 | 0 | 67 | 62 | 27 | 37 |
| UICC II, % | | 32 | 0 | 11 | 19 | 20 | 32 |
| UICC III, % | | 25 | 0 | 22 | 14 | 53 | 23 |
| UICC IV, % | | 0 | 100 | 0 | 5 | 0 | 8 |
| adjuvant therapy | | 18/53 | — | 7/13 | 121/261 | 15/27 | 161/375 |
| follow up, median [d] | | 761 | 375 | 711 | 723 | 707 | 722 |
| age, median (range) [y] | 60 (27-66) | 67 (23-86) | 66 (37-78) | 70 (49-76) | 67 (23-85) | 65 (52-76) | 67 (23-86) |
| sex, m/f | 37/14 | 35/18 | 15/3 | 8/5 | 169/92 | 17/10 | 244/131 |
| S100A4 transcript levels | | | | | | | |
| S100A4, median | 0.219 | 0.425 | 0.578 | 0.532 | 0.374 | 0.595 | 0.399 |
| P, vs tumor-free volunteers | | <0.0001 | <0.0001 | <0.0001 | <0.0001 | <0.0001 | <0.0001 |

Colon cancer patients characteristics and S100A4 transcript levels

| | | colon cancer patients | | | | | |
|---|---|---|---|---|---|---|---|
| | | UICC | UICC | meta- | follow-up | follow-up | |
| | tumor-free | stages | stage | chronous | w/o | with | all cancer |
| | volunteers | I-III | IV | matastasis | metastasis | metastasis | patients |
| Donor characteristics | | | | | | | |
| samples, n | 51 | 13 | 10 | 5 | 145 | 12 | 185 |
| UICC I, % | | 47 | 0 | 0 | 25 | 0 | 23 |
| UICC II, % | | 38 | 0 | 60 | 45 | 68 | 44 |
| UICC III, % | | 15 | 0 | 40 | 28 | 31 | 26 |
| UICC IV, % | | 0 | 100 | 0 | 2 | 0 | 7 |
| adjuvant therapy | | 2/13 | — | 1/5 | 48/145 | 5/12 | 56/185 |
| follow up, median [d] | | 726 | 181 | 274 | 742 | 695 | 729 |
| age, median (range) [y] | 60 (27-86) | 64 (51-76) | 66 (37-78) | 64 (49-76) | 68 (37-83) | 65 (54-67) | 67 (37-83) |
| sex, m/f | 37/14 | 10/3 | 6/4 | 1/4 | 86/59 | 9/3 | 112/73 |
| S100A4 transcript levels | | | | | | | |
| S100A4, median | 0.219 | 0.347 | 0.6825 | 0.631 | 0.403 | 0.67 | 0.434 |
| P, vs tumor-free volunteers | | 0.0055 | <0.0001 | 0.0023 | <0.0001 | <0.0001 | <0.0001 |

Rectal cancer patients characteristics and S100A4 transcript levels

| | | rectal cancer patients | | | | | |
|---|---|---|---|---|---|---|---|
| | | UICC | UICC | meta- | follow-up | follow-up | |
| | tumor-free | stages | stage | chronous | w/o | with | all cancer |
| | volunteers | I-III | IV | metastasis | metastasis | metastasis | patients |
| Donor characteristics | | | | | | | |
| samples, n | 51 | 40 | 11 | 8 | 116 | 15 | 190 |
| UICC I, % | | 43 | 0 | 67 | 62 | 27 | 51 |
| UICC II, % | | 32 | 0 | 11 | 19 | 20 | 21 |
| UICC III, % | | 25 | 0 | 22 | 14 | 53 | 19 |
| UICC IV, % | | 0 | 100 | 0 | 5 | 0 | 9 |
| adjuvant therapy | | 16/40 | — | 6/8 | 73/116 | 10/15 | 105/190 |
| follow up, median [d] | | 762 | 794 | 751 | 705 | 744 | 713 |
| age, median (range) [y] | 60 (27-86) | 67 (23-86) | 65 (62-73) | 70 (53-76) | 67 (23-85) | 66 (52-76) | 67 (23-86) |
| sex, m/f | 37/14 | 25/15 | 9/2 | 7/1 | 83/33 | 8/7 | 132/58 |
| S100A4 transcript levels | | | | | | | |
| S100A4, median | 0.219 | 0.4375 | 0.474 | 0.319 | 0.3645 | 0.563 | 0.38 |
| P, vs tumor-free volunteers | | <0.0001 | 0.0054 | 0.0335 | <0.0001 | 0.0017 | <0.0001 |

Gastric cancer patients characteristics and S100A4 transcript levels

| | | gastric cancer patients | | | | | |
|---|---|---|---|---|---|---|---|
| | | UICC | UICC | meta- | follow-up | follow-up | |
| | tumor-free | stages | stage | chronous | w/o | with | all cancer |
| | volunteers | I-IV(M0) | IV(M1) | metastasis | metastasis | metastasis | patients |
| Donor characteristics | | | | | | | |
| samples, n | 51 | 11 | 10 | 6 | 60 | 4 | 91 |
| UICC I, % | | 45 | 0 | 50 | 50 | 0 | 45 |
| UICC II, % | | 21 | 0 | 0 | 30 | 50 | 25 |
| UICC III, % | | 18 | 0 | 17 | 17 | 25 | 15 |
| UICC IV, % | | 10 | 100 | 33 | 3 | 25 | 15 |
| adjuvant therapy | | 1/11 | — | 2/6 | 0 | 0 | 3/61 |
| follow up, median [d] | | 693 | 111 | 62 | 735 | 590 | 712 |
| age, median (range) [y] | 60 (27-86) | 63 (42-79) | 63 (57-77) | 71 (38-83) | 63 (40-86) | 63 (49-74) | 63 (38-86) |
| sex, m/f | 37/14 | 9/2 | 9/1 | 5/1 | 40/20 | 4/0 | 67/24 |
| S100A4 transcript levels | | | | | | | |
| S100A4, median | 0.219 | 0.509 | 0.5075 | 0.632 | 0.384 | 0.5635 | 0.411 |
| P, vs tumor-free volunteers | | <0.0001 | 0.005 | 0.0032 | <0.0001 | 0.0188 | <0.0001 |

TABLE 2
Sensitivity and specificity for S100A4 transcripts levels in plasma of colorectal,
colon, rectal, and gastric cancer patients with newly diagnosed primary tumors
without and with metastasis, and with metachronous metastasis

| Cancer | samples, n | cut-off | | sensitivity, % | specificity, % |
|---|---|---|---|---|---|
| Colorectal | | | | | |
| Test set | 44 | 0.358 | | 73 | 82 |
| Validation set | 43 | 0.358 | colorectal cancer patients' test set | 68 | 82 |
| All | 87 | 0.358 | colorectal cancer patients' test set | 71 | 82 |
| Colon | 28 | 0.358 | colorectal cancer patients' test set | 68 | 82 |
| Colon | 28 | 0.232 | optimal cut-off for colon cancer cohort | 96 | 59 |
| Rectal | 59 | 0.358 | colorectal cancer patients' test set | 72 | 82 |
| Rectal | 59 | 0.346 | optimal cut-off for rectal cancer cohort | 74 | 82 |
| Gastric | 27 | 0.289 | optimal cut-off for gastric cancer cohort | 90 | 71 |

1. A method for diagnosis and/or prognosis of a gastrointestinal cancer, a stage of gastrointestinal cancer or a gastrointestinal cancer free stage comprising:
(a) providing a basal transcript level or a transcript level of a stage of gastrointestinal cancer;
(b) measuring a S100A4 transcript level in a body fluid of a target subject;
making a determination as to whether or whether not there is a significant difference in S100A4 transcript level between (a) and (b); and
making a diagnosis and/or prognosis as to whether or whether not the target subject suffers from a gastrointestinal cancer or a certain stage thereof or is at a gastrointestinal cancer free stage based on said determination.

2. The method of claim 1, wherein transcript levels of two or more stages of gastrointestinal cancer are provided in (a).

3. The method of claim 2, wherein the diagnosis and/or prognosis of the target subject is as to whether or not the target subject has or has not one of said two or more stages of gastrointestinal cancer and wherein said determination as to whether or not there is a significant difference comprises or consists essentially of comparing transcript levels of (a) with that measured in (b).

4. The method of claim 1, wherein RT-PCR is used for said measuring of said S100A4 transcript level in said target subject.

5. The method of claim 1, wherein S100A4 transcription level(s) in (a) is/are provided from measurements, preferably measurements obtained via RT-PCR, of one or more previous cohorts.

6. The method of claim 5, wherein said transcript level(s) is/are provided for one or more mRNA calibrators used for said measurements in said one or more previous cohorts.

7. The method of claim 1, wherein said S100A4 transcript level(s) in (a) is/are provided from measurements of a contemporary population of the target subject.

8. A kit for transcript quantification of S100A4 in a target subject comprising, in different containers:
a tool for quantitatively determining S100A4 transcript levels in a body fluid; and
values for a basal S100A4 transcript level or instructions of how to obtain a basal transcription level of S100A4 transcripts.

9. The kit of claim 8, further comprising a calibrator for quantitatively determining S100A4 transcript levels in a body fluid.

10. The kit of claim 9, wherein calibrator corresponds to the calibrator used to determine said values for said basal S100A4 transcript level.

11. The kit of claim 8, wherein said tool comprises:
(a) primers for reverse transcribing S100A4 transcripts in a sample;
(b) a reverse transcriptase for producing cDNA of said S100A4 transcripts;
(c) DNA primers for amplifying said cDNA, and
(d) a DNA polymerase for extending said primers.

12. The kit of claim 8, further comprising values or instruction of how to obtain values for a transcription level representative of different stages of gastrointestinal cancers, in particular representative for patients newly diagnosed with a primary tumor, for patients newly diagnosed with a primary tumor together with synchronous metastases; for patients who already underwent RO-surgery of the primary cancer and were newly diagnosed with metachronous metastases; for follow-up patients without metastases; for follow-up patients with metastases; follow-up gastrointestinal cancer patients with or without metastases or any combination of the same and preferably a calibrator that was used in obtaining said values.

13. The kit of claim 8, wherein the values are provided as part of the kit.

14. The kit of claim 8, wherein the instructions include information as to where those values can be obtained.

15. The kit of kit of claim 8, further comprising instructions of what values need to be compared for the prognosis and/or diagnosis of gastrointestinal cancer, its stages and/or a gastrointestinal cancer free stage.
